(19) United States
(12) Patent Application Publication
(10) Pub. No.: US 2022/0215929 A1
(43) Pub. Date: Jul. 7, 2022

(54) PET HEALTH MONITORING SYSTEM

(71) Applicant: Full Life Wellness, LLC, Phoenix, AZ (US)

(72) Inventors: William Levy, Phoenix, AZ (US); Scott Eller, Phoenix, AZ (US)

(73) Assignee: Full Life Wellness, LLC, Phoenix, AZ (US)

(21) Appl. No.: 17/568,548

(22) Filed: Jan. 4, 2022

Related U.S. Application Data
(60) Provisional application No. 63/133,583, filed on Jan. 4, 2021.

(51) Int. Cl. G16H 20/60; G16H 20/30; G16H 40/67; A61B 5/00

(52) U.S. Cl. CPC G16H 20/60; G16H 20/30; A61B 2503/40; A61B 5/6898; G16H 40/67

(57) ABSTRACT

A system enables monitoring and improving one or more of the habits, diet, exercise, and activities related to a pet's health and includes a network having one or both of a smartphone and a personal computer connected to the network, a food preparation device connected to the network, and a server controlled by a system manager and connected to the network.

### FIELD

The present invention is related to pet parenting. More specifically, the invention is related to a system for monitoring and improving the habits, diet, exercise, activities, and other facets of a pet's health.

### BACKGROUND

A common and age-old problem is that pets are prone to overeat, live an inactive lifestyle, and do other things which may shorten their already too short lives. Without an incentive to do otherwise, pet's parents are prone to overfeed and pamper their pets, which adds to that shortening.

There is the need, and it may be an objective of the invention, to provide a system to improve the habits, diet, exercise, activities, and other facets of a pet's health.

There also is the need, and it may be an objective of the invention, to provide such a system including a food preparation device capable of providing a healthful amount of food or a healthful type.

There also is the need, and it may be an objective of the invention, to provide such an device which is connectable to a network for remote assistance.

There also is the need, and it may be an objective of the invention, to provide such a network which is wireless.

There also is the need, and it may be an objective of the invention, to provide such a system which includes a smartphone app capable of communicating through the network and adapted to receive input from the pet's parent and to provide a meal plan and/or health plan according thereto to the pet's parent.

There also is the need, and it may be an objective of the invention, to provide such a smartphone app capable of providing feedback from the device to the network.

Further needs and objectives will become apparent upon perusal of the disclosure herein.

### SUMMARY

The herein disclosed exemplary system, including the herein disclosed exemplary Device, combines the ability to monitor and improve the habits, diet, exercise, activities, and other facets of a pet's health, with the ability to allow the pet's parent to control the same according to a dietary plan based on the pet's specific needs.

The device may be capable of preparing the pet a healthful amount of food or a healthful type, according to a calculated meal plan. The device may be connectable to a network for data capture and exchange. The network may be wireless.

The system may include a smartphone app capable of communicating through the network and adapted to receive pet-specific information from the pet's parent, process that information into a health plan including a meal plan, and to provide the meal plan and meals according thereto to the pet's parent for preparing through the device. The smartphone app may be capable of providing feedback from the Device to the pet's parent.

Further features of the invention will become apparent upon perusal of the disclosure herein.

### BRIEF DESCRIPTION OF THE DRAWINGS

Exemplary embodiments of systems for monitoring and improving the habits, diet, exercise, activities, and other facets of a pet's health in accordance with or useful in practicing the invention is shown in the accompanying Drawings, of which;

FIG. 1 is a schematic view of a first system;

FIG. 2 is a view of the food preparation device of the system of FIG. 1 being loaded with food cups;

FIG. 3 is a view of the food preparation device of the system of FIG. 1 in its loaded and ready state;

FIG. 4 is a cross-sectional view of the food preparation device of the systems of FIG. 1 in its loaded and ready state;

FIG. 5 is a view showing the underside of the food cups of FIG. 2;

FIG. 6 is a flow diagram of the operation of the system of FIG. 1; and

FIG. 7 is a flow diagram of the operation of a second system.

### DESCRIPTION OF EXEMPLARY EMBODIMENTS

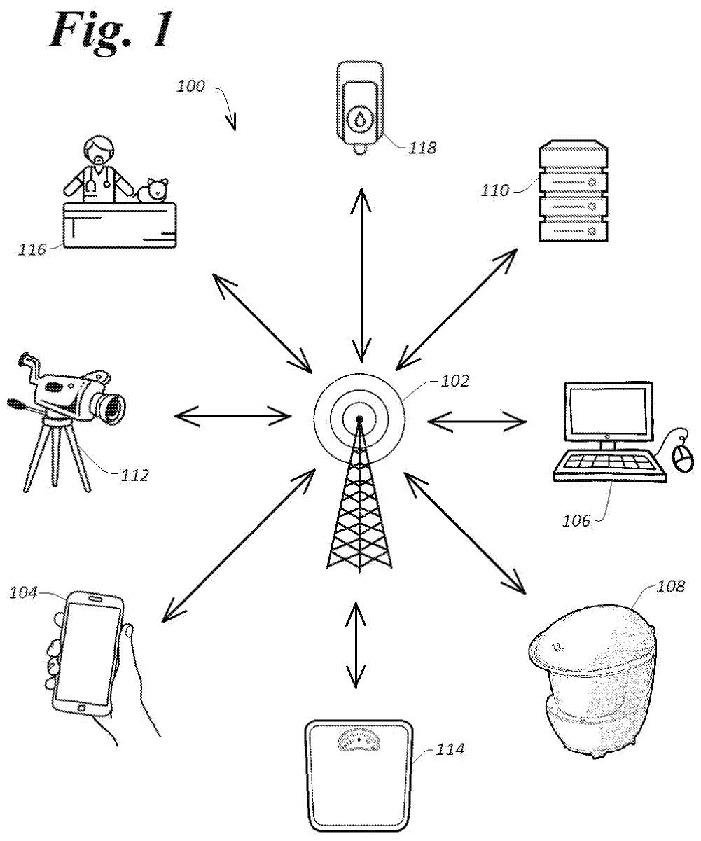

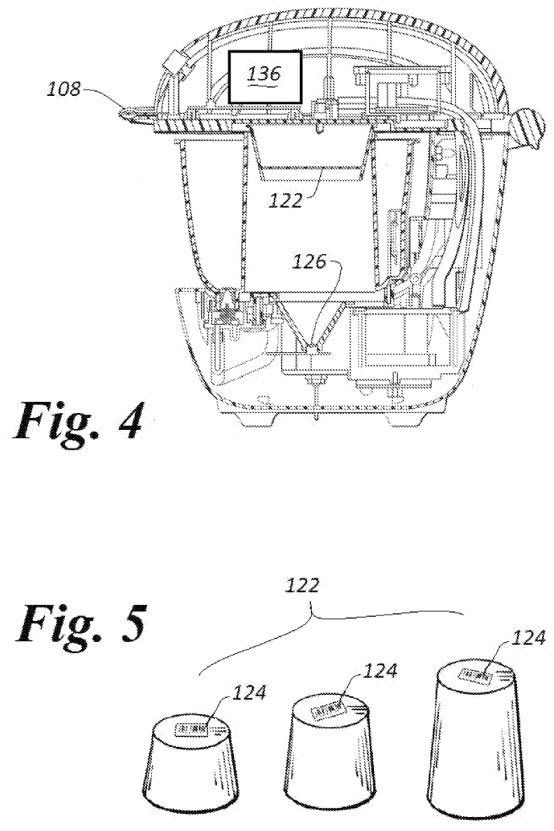

Referring first to FIGS. 1 through 6, a first exemplary system 100 for monitoring and improving the habits, diet, exercise, activities, and other facets of a pet's health is shown. The system includes some or all of the following components; a wireless or Internet network 102, a smartphone 104, a personal computer 106, a food preparation device 108, a server 110, an activity monitor 112, electronic scale 114, veterinarian service 116, and medicine dispenser 118.

While FIG. 1 shows an icon of a wireless antenna to symbolize the network, the icon may also be interpreted to represent a hardwired Internet network or such.

Regarding the food preparation device, this application claims benefit to U.S. patent Ser. No. 10/966,409 issued on 6 Apr. 2021, and to U.S. Application Ser. No. 17/150,758 filed on 15 Jan. 2021, and 16/175,145 filed on 30 Oct. 2018. The descriptions of the food preparation devices therein are incorporated herein in their entireties by reference and are not repeated herein for brevity, but any reference made herein to a food preparation device should be considered fully disclosed by such incorporation by reference excepting that the version of the device of the present application is equipped with a networking interface 136 for use in the present system. And while the icon of the food preparation device in FIG. 1 shows the food preparation device of patent Ser. No. 10/966,409, it should be understood that any pet food preparation device capable fulfilling the role required by the system could be substituted therefore.

The smartphone is carried by the pet's parent and loaded with a smartphone app 128 adapted to both allow receive and transmit information about the pet and its care through the network.

The optional personal computer (PC) may also be loaded with the same app, a more PC-specific program, or may access the system through a system website via the Internet.

The server may be located where convenient and practical, such as at a server hosting facility or at a system managing headquarters. The server is ultimately controlled and operated by a system manager or its assigns, who may simultaneously employ the server to manage many equivalent pet health monitoring and improving systems.

The electronic scale is capable of accurately weighing the pet, and is equipped with network access capabilities, either through the food preparation device or in parallel therewith, so that it can communicate wirelessly or through the Internet to one or more of the other components of the system. The scale thereby transmits the recorded weight to the network.

The medication dispenser is of the type that releases a preloaded medication or vitamin dosage so that it becomes available for the pet's consumption. This dispenser also has the ability for network access, either through the food preparation device or in parallel therewith, so that it can communicate wirelessly or through the Internet to one or more of the other components of the system. The medication dispenser thereby reports the dispensed medication to the network.

The veterinarian service may be an actual private veterinarian of the parent's choosing who has access and capability to use the system or it may be a veterinarian service provided by the system manager, or it may be an autonomous virtual veterinarian.

The activity monitor may be as simple as a security camera which watches the pet and either records or provides a live stream of its activities which can be viewed by the pet's parent. Or it may be a more sophisticated monitoring system subsystem such as one which includes a base monitoring device and a pet-worn collar connected together by Bluetooth or such. The collar might include features such as a GPS tracker, a pace monitor, a heartbeat monitor, a thermometer, or means of measuring and reporting numerous other aspects of the pet's health and activities that can be transmitted to the network, either through the food preparation device or independently, such that they are available to the parent, veterinarian service, app and system.

The information may be transmittable, through all components or some, through the food preparation device or directly to the server via the wireless or Internet connection, and all information placed onto the network by any of the components is available for use and viewing by any other of the other components which may benefit from the information.

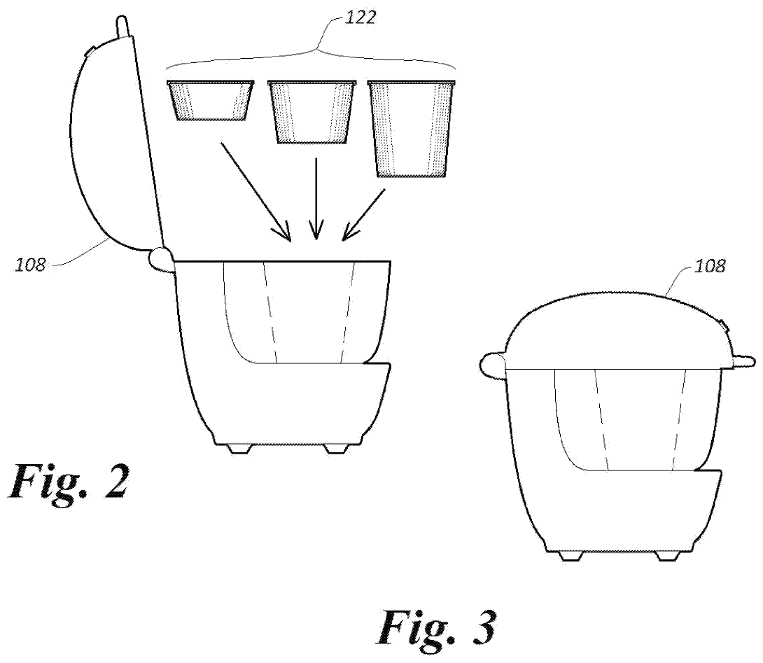

As explained in patent Ser. No. 10/966,409 and as depicted in FIGS. 2, 4, and 5, the food preparation device may be adapted to receive, identify, and certify cups of food 122, . . . each having a barcode 124 on its underside which is read by a scanner 126 in the food preparation device and used to identify and verify the volume and ingredients of the food they contain. These food cups are available from the system manager or its assigns in a variety of sizes and with a variety of food ingredients. The food ingredients may be formulated according to pet age, breed, health, weight and/or dietary needs or restrictions that had been provided by the parent and/or the components of the system.

Once the parent has set up a subscription with the system manager, received the feeding machine from the system manager or one of its assigns, loaded the app into a smartphone or through the PC, and reported the pet-specific information, and had received the health plan, including at least a meal plan, and the pet specific foods indicated thereby, the parent loads the upcoming meal into the food preparation device. The device may automatically prepare the meal and/or may alert the parent by alarm and/or notification through the app, at the times that meals should be prepared and served.

The app will automatically track and monitor the feeding portion by verifying the meal that was prepared and will transmit that through the network.

The other devices may alert the parent by alarm and/or notification through the app, as their function should be used or activated according to the remainder of the health plan.

The app may cause the activity monitor to record and transmit the desired activities and health readings and transmit them to the network.

The veterinarian service (if employed) may use from the information things such as food consumption, weight, and activity to adjust or to advise adjustment of related portions of the health plan.

The algorithm will receive such advice, and employ it with other received information to adjust the health plan, including at least the meal plan, as needed, and will transmit that to the network and to at least the app or website.

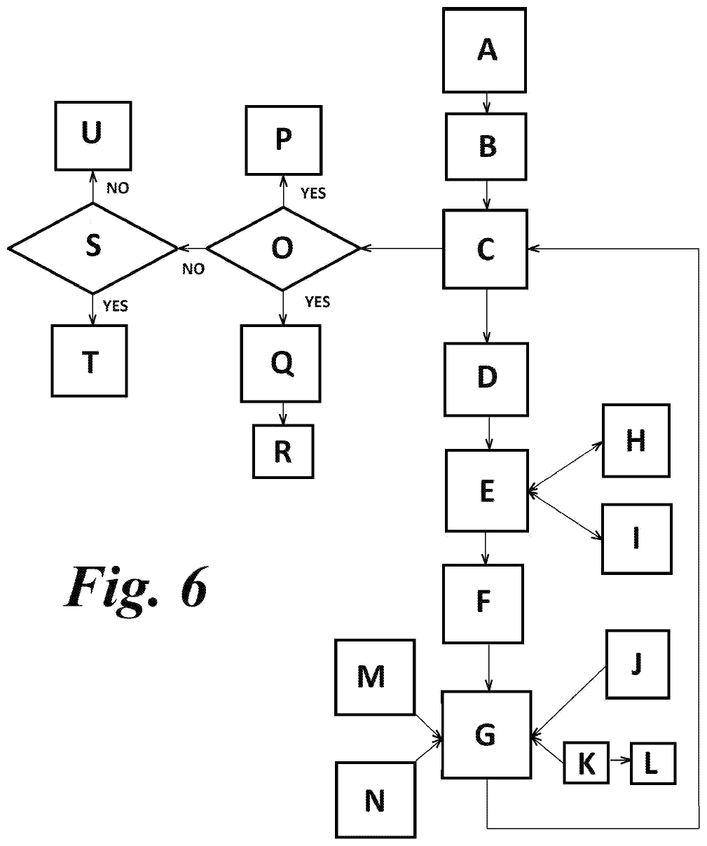

Referring to FIG. 6, there is shown a flow chart for the system, in which each block is identified by a letter corresponding to the following list;
A. After having purchased a subscription from the system manager and downloaded the app or created an Internet account, the pet's parent inputs the pet's breed, weight, age, health history, and usual activity level information into the smartphone app or website.
B. The app or website communicates that information to an algorithm in the server.
C. The algorithm calculates the pet's caloric need, nutritional need, medicinal need, and activity need based on the information and makes a daily plan recommendation to the parent, including at least a meal plan recommendation.
D. Once the meal plan recommendation is accepted by the parent, the food preparation device, meals, and other devices (if employed) are sent to the parent.
E. The food preparation device is connected to a wi-fi network via Bluetooth and a smart phone app onboarding method, or is connected via the Internet through the PC. The other devices (if employed), are connected either directly or through the food preparation device, and activated.
F. The food preparation device's bar code reader recognizes each meal inserted into the food preparation device via the bar code on the bottom of the meal cup and begins the appropriate cooking cycle.
G. The food preparation device communicates identification and preparation of the meal to the network, and the server's algorithm uses that to monitor and adjust caloric and nutritional intake, and to produce a maintenance report which it transmits to the network, including to at least the app and/or the website.
H. The electronic scale (if employed) is connected to the network either directly or through the food preparation device and communicates the pet's weight to the network, and the server's algorithm uses that to monitor and adjust caloric intake, and to produce the maintenance report.
I. The activity monitor (if employed) is connected to the network either directly or through the food preparation device and communicates the pet's activities to the network, and the server's algorithm uses that to monitor and adjust things like caloric intake and medication, and to produce the maintenance report. The activity monitor may issue an “Exercise Pet Now” alert when appropriate, through the app and/or by alarm.
J. The food preparation device may issue a “Feed Pet Now” alert when appropriate, through the app and/or by alarm.
K. The Scale may issue a “Weigh Pet Now” alert when appropriate, through the app and/or by alarm.
L. The alerts may be issued via email, text, or smart phone app notifications and/or by sound/visual alarms by the applicable devices.
M. Is there a need to adjust caloric intake?
N. New or existing daily plan, weight target, and/or recommended activities may be formulated by the veterinarian service (if employed).
O. The new or existing daily plan, weight target, and/or recommended activities may be uploaded by the veterinarian service (if employed) to a tele-health portal.
P. The tele-health portal may make the new or existing daily plan, weight target, and/or recommended activities available to the parent via the app or website.
Q. Are the food preparation device, scale, and/or activity monitor functioning properly?
R. A new food preparation device, scale, and/or activity monitor is sent to the parent.
S. The parent keeps using the existing food preparation device, scale, and/or activity monitor.

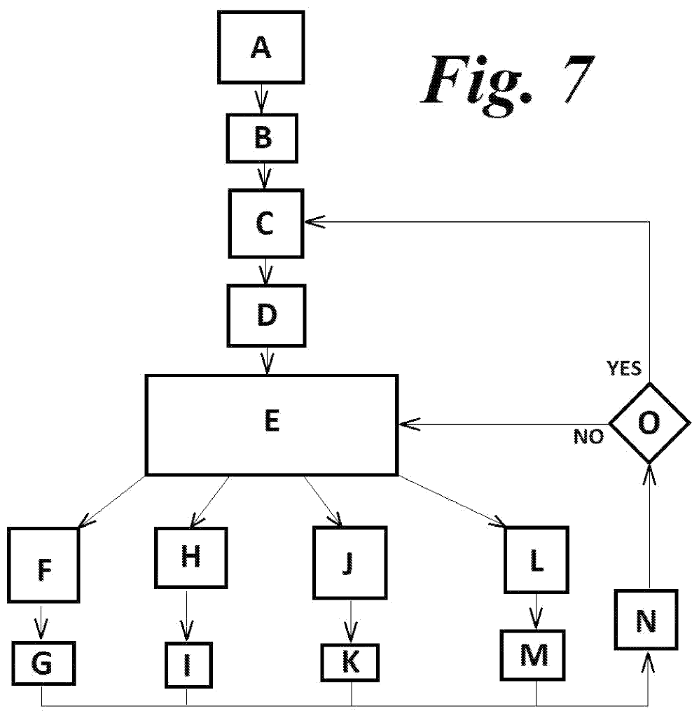

Referring to FIG. 7, there is shown a flow chart for an alternate system, in which each block is identified by a letter corresponding to the following list;
A. After having purchased a subscription from the system manager and downloaded the app or created an Internet account, the pet's parent inputs the pet's breed, weight, age, health history, and usual activity level information into the smartphone app or website.
B. The app or website communicates that information to an algorithm in the server.
C. The algorithm calculates the pet's health plan, including at least a meal plan, based on the information and makes a plan recommendation to the parent, including at least a meal plan recommendation.
D. The server communicates the health plan to the network.
E. The app issues alerts to the parent to remind of feeding, medication, health monitoring and exercise, as due. The app provides a report received from the algorithm and including at least pet feeding history, health history, weight history, and medication history on demand through the app and/or website.
F. The food preparation device issues a “Feed Now” alert through the app and/or by the device.
G. The food preparation device identifies the meal inserted and reports that to the network.
H. The scale (if employed) issues a “Weigh Now” alert through the app and/or by the scale or food preparation device.
I. The parent weighs the pet on the scale (if employed) and the scale reports the measured weight to the network.
J. The activity monitor (if employed) issues a “Monitor Now” alert through the app and/or the monitor or the device.
K. The parent measures recommended health conditions of the pet using the activity monitor (if employed) and the monitor reports those to the network.
L. The medication dispenser (if employed) issues a “Medicate Now” alert through the app and/or the dispenser or the food preparation device.
M. The medication dispenser dispenses and repots to the network.
N. All events, results, and information are communicated wirelessly or via the Internet to the server's algorithm.
O. In violation of the health plan?

It is anticipated that some of the steps and functions of each of the two systems above may be removed or may be added from one to the other.

While the forgoing provides exemplary embodiments are meant to demonstrate key aspects and advantages of the invention, it should be understood that the invention is not meant to be limited thereto, and the invention should be limited only by the following claims, including all equivalents thereto.

We claim:

1. A system for monitoring and improving one or more of the habits, diet, exercise, and activities related to a pet's health comprising:
a network having one or both of a smartphone and a personal computer connected to the network,
a food preparation device connected to the network, and
a server controlled by a system manager and connected to the network;
wherein one or both of an app of the smartphone and an Internet website accessed by the personal computer cooperates with an algorithm within the server through the network to create a meal plan; the system manager provides a food preparation device and meals according to the meal plan, and the food preparation device prepares the meals and reports preparation thereof to the algorithm.

2. The system of claim 1 wherein the food preparation device comprises a meal code reader connected to the network and adapted to recognize and verify contents and volume of each of the meals.

3. The system of claim 2 wherein each of the one or more meals comprises a meal code recognizable by the meal code reader.

4. The system of claim 3 wherein the meal code is specific to the contents and volume of each of the meals, enabling the food preparation device to confirm that the meal to be prepared is one of those provided and is according to the meal plan.

5. The system of claim 4 wherein the meals are provided in meal cups and the food preparation device is adapted to receive each of the meal cups such that its meal code is visible to the meal code reader.

6. The system of claim 5 wherein the food preparation device is adapted to receive meal cups of various volumes such that the meal code of each is visible to the meal code reader.

7. The system of claim 6 further comprising a weighing scale connected to the network; and wherein the weighing scale reports a measurement of the pet's weight to the algorithm.

8. The system of claim 7 further comprising a veterinarian service connected to the network; and wherein the veterinarian service provides veterinarian advice to one or all of the app, the website and algorithm.

9. The system of claim 8 wherein the veterinarian advice is derived from the information received from the network.

10. The system of claim 9 wherein the information received from the network includes one or more of the pet's breed, age, weight history, diet history, activity history, medication history, and health history.

11. A method for monitoring and improving one or more of the habits, diet, exercise, and activities related to a pet's health comprising:
providing a network, a server having an algorithm connected to the network, and one or both from the group including a smartphone app and a website connectable to the network;
providing one or both of a smartphone connected to the network and a personal computer connected to the network.
performing one or both of the steps including installing the app on the smartphone and accessing the website with the personal computer;
inputting information over the network to the algorithm including one or more of the pet's breed, weight, age, health history, and usual activity level into one or both of the app and the website;
calculating a plan from the information including one or more of the pet's caloric need, nutritional need, medicinal need, and activity need by the algorithm;
providing a food preparation device and one or more meals according to the plan;
connecting the food preparation device to the network;
verifying and preparing the one or more meals by the food preparation device;
reporting verification and preparation to the algorithm over the network by the food preparation device;
recalculating the plan according to the reported preparation and reporting historical meal preparation data to one or both of the app and the website.

12. The method of claim 11 further comprising employing a meal code reader of the food preparation device to recognize and verify contents and volume of each of the or more meals.

13. The method of claim 12 wherein each of the one or more meals comprises a meal code recognizable by the meal code reader.

14. The method of claim 13 wherein the meal code is specific to the contents and volume of each of the meals, enabling the food preparation device to confirm that the meal to be prepared is one of those provided and is according to the meal plan.

15. The method of claim 14 further comprising providing the one or more meals in meal cups wherein the food preparation device is adapted to receive each of the meal cups such that its meal code is visible to the meal code reader.

16. The method of claim 15 wherein the food preparation device is adapted to receive meal cups of various volumes such that the meal code of each is visible to the meal code reader.

17. The method of claim 16 further comprising:
employing a scale connected to the network to recognize and verify adherence to weight-maintenance aspects of the plan.

18. The method of claim 17 further comprising:
employing an activity monitor connected to the network to recognize and verify adherence to activity aspects of the plan.

19. The method of claim 18 further comprising:
employing a veterinarian service connected to the network to provide one or both of dietary and activity advice for the pet.

20. The method of claim 19 wherein dietary and activity advice is derived from the information received from the network.
